(19) United States
(12) Patent Application Publication
(10) Pub. No.: US 2018/0256038 A1
(43) Pub. Date: Sep. 13, 2018

(54) SYSTEM, CATHETER, AND METHOD FOR CALCULATING CORRECTED FRACTIONAL FLOW RESERVE

(71) Applicant: Medtronic Vascular, Inc., Santa Rosa, CA (US)

(72) Inventor: Jignesh Shah, Sunnyvale, CA (US)

(21) Appl. No.: 15/453,637

(22) Filed: Mar. 8, 2017

(51) Int. Cl. A61B 5/026; A61B 5/0215; A61B 5/01; A61B 5/00

(52) U.S. Cl. CPC A61B 5/026; A61B 5/6852; A61B 5/01; A61B 5/02158

(57) ABSTRACT

A catheter includes an elongate shaft, a pressure sensor, and a thermistor. The elongate shaft defines a guidewire lumen. The pressure sensor is coupled to a distal portion of the elongated shaft such that the pressure sensor senses a blood pressure distal of a stenosis. The thermistor is coupled to the distal portion of the elongate shaft such that the thermistor measures blood flow velocity distal of the stenosis. The pressure sensor and thermistor are in communication with a processor. The processor calculates a corrected Fractional Flow Reserve including a correction based on blood flow velocity.

### FIELD OF THE INVENTION

The present invention relates to systems and methods for calculating a Fractional Flow Reserve. More particularly, the present invention relates to calculating a corrected Fractional Flow Reserve with a catheter, wherein the corrected Fractional Flow Reserve accounts for blood flow velocity.

### BACKGROUND OF THE INVENTION

The severity of a stenosis or lesion in a blood vessel may be assessed by obtaining proximal and distal pressure measurements relative to the given stenosis and using those measurements for calculating a value of a Fractional Flow Reserve (FFR). FFR is defined as the ratio of a first or distal pressure P_{d} measured on the distal side of the lesion to a second or proximal pressure Pₐ measure on the proximal side of the lesion, usually within the aorta. Conventionally, a sensor is placed on a distal portion of a guidewire or FFR wire to obtain the distal pressure P_{d}, while an external pressure transducer is fluidly connected via tubing to a guide catheter for obtaining the proximal, or aortic (AO) pressure Pₐ. Calculation of the FFR value provides a lesion specific index of the functional severity of the stenosis in order to determine whether the blockage limits blood flow within the vessel to an extent that treatment is needed. An optimal or normal value of FFR in a healthy vessel is 1.00, while values less than about 0.80 are generally deemed significant and in need of an interventional treatment. Common interventional treatment options include balloon angioplasty and/or stent implantation.

If an interventional treatment is required, the interventional device, such as a balloon catheter, is tracked over a guide wire to the site of the lesion. Conventional FFR wires generally are not desired by clinicians to be used as guide wires for such interventional devices. Accordingly, if an intervention treatment is required, the clinician generally removes the FFR wire, inserts a conventional guide wire, and tracks the interventional device to the treatment site over the conventional guide wire.

To address this concern, efforts have been made to utilize catheters to take pressure measurements for calculating FFR. Using a catheter, a clinician may use a preferred guidewire for tracking the FFR catheter to the site of the lesion. If an interventional treatment is required, the guidewire used with the catheter may remain in situ and the interventional device may be tracked over the existing guidewire to the site of the lesion.

However, some deviation from FFR values calculated using an FFR guidewire may be introduced into the distal blood pressure P_{d} measured distal of the lesion if a cross-sectional size of the portion of the measurement device (catheter) that crosses the lesion is larger than a conventional FFR guidewire. Further, the distal blood pressure P_{d}, and hence FFR value, is affected by blood flow rate or velocity. In other words, for the same stenosis, different FFR values may be obtained depending on the blood flow velocity distal of the stenosis. Because decisions regarding intervention have been based on FFR values taken using FFR guidewires, there is a need to correlate or correct FFR values calculated using catheters to FFR values taken using guidewires. Accordingly, correction factors for correlating FFR values taken using a catheter have been developed to account for size differences between FFR catheters and FFR guidewires. However, there remains a need to accurately account for blood flow velocity.

### BRIEF SUMMARY OF THE INVENTION

Embodiments hereof relate to a catheter including an elongate shaft, a pressure sensor, and a thermistor. The elongate shaft defines a guidewire lumen. The pressure sensor is coupled to a distal portion of the elongate shaft such that a pressure-sensing surface of the pressure sensor faces outside an outer surface of the distal portion of the elongate shaft. The thermistor is coupled to the distal portion of the elongate shaft such that a surface of the thermistor faces outside the outer surface of the distal portion of the elongate shaft. The pressure sensor senses a pressure of blood distal of a stenosis in a blood vessel. The pressure sensor and thermistor are in communication with a processor. The thermistor measures blood flow velocity distal of the stenosis such that the processor may calculate a corrected Fractional Flow Reserve including a correction based on blood flow velocity.

Embodiments hereof also relate to a system for calculating a corrected Fractional Flow Reserve associated with a stenosis in a blood vessel. The system includes a pressure sensor, a pressure-sensing device, a thermistor, and a processing device. The pressure sensor is placed within a blood vessel to measure a distal pressure distal of the stenosis. The pressure-sensing device measures a proximal pressure proximal of the stenosis. The thermistor is placed within the blood vessel to measure blood flow velocity distal of the stenosis. The processing device is in communication with the pressure sensor, the pressure-sensing device, and the thermistor. The processing device calculates a Fractional Flow Reserve based on the distal pressure relative to the proximal pressure. The processing device calculates or selects a transfer function including a flow velocity correction based on the measured blood flow velocity.

Embodiments hereof also relate to a method for calculating a corrected Fractional Flow Reserve in a vessel. A catheter is delivered to a distal side of a stenosis of the vessel. The catheter includes a distal pressure sensor and a thermistor. The blood flow velocity within the vessel is measured utilizing the thermistor. A transfer function including a blood flow velocity correction based on the measured blood flow velocity is calculated or selected. A pressure distal of the stenosis is measured. A pressure proximal of the stenosis is measured. The corrected Fractional Flow Reserve is calculated using the measured proximal and distal pressures and application of the transfer function.

### BRIEF DESCRIPTION OF DRAWINGS

FIG. 1 is an illustration of a system for calculating a corrected Fractional Flow Reserve (FFR_{CORR}) with a catheter thereof shown within a vessel including a lesion, in accordance with an embodiment hereof.

FIG. 2 is a partial longitudinal cross-section illustration of the system and catheter of FIG. 1.

FIG. 3 is a cross-sectional illustration of an embodiment of a proximal portion of the catheter, taken along line 3-3 of FIG. 2.

FIG. 4 is a cross-sectional illustration of an embodiment of a distal portion of the catheter, taken along line 4-4 of FIG. 2.

FIG. 5 is a close-up top cutaway illustration of an embodiment of the distal portion of the catheter of FIG. 2.

FIG. 6 is a close-up side cutaway illustration of the distal portion of the catheter of FIG. 2.

FIG. 7 is a schematic diagram of an embodiment of a Wheatstone bridge with a thermistor as a leg of the Wheatstone bridge.

FIG. 8 is a graph illustrating FFR measured using a 0.024 inch FFR catheter compared to FFR measured using a 0.014 inch FFR wire for different flow rates and different lesions.

FIG. 9 is a block diagram of an embodiment of a method for calculating corrected Fractional Flow Reserve (FFR_{CORR}) using the system of FIG. 1.

### DETAILED DESCRIPTION OF THE INVENTION

Specific embodiments of the present invention are now described with reference to the figures, wherein like reference numbers indicate identical or functionally similar elements. The terms “distal” and “proximal”, when used in the following description to refer to a catheter or delivery system are with respect to a position or direction relative to the treating clinician. Thus, “distal” and “distally” refer to positions distant from, or in a direction away from the treating clinician, and the terms “proximal” and “proximally” refer to positions near, or in a direction toward the clinician. The terms “distal” and “proximal” used in the following description to refer to a vessel or a lesion are used with reference to the direction of blood flow. Thus, “distal” and “distally” refer to positions in a downstream direction with respect to the direction of blood flow, and the terms “proximal” and “proximally” refer to positions in an upstream direction with respect to the direction of blood flow.

The following detailed description is merely exemplary in nature and is not intended to limit the invention or the application and uses of the invention. Although the description of the invention is in the context of treatment of blood vessels such as the coronary arteries, the invention may also be used in any other body passageways where it is deemed useful such as but not limited to peripheral arteries, carotid arteries, renal arteries, and/or venous applications. Furthermore, there is no intention to be bound by any expressed or implied theory presented in the preceding technical field, background, brief summary or the following detailed description.

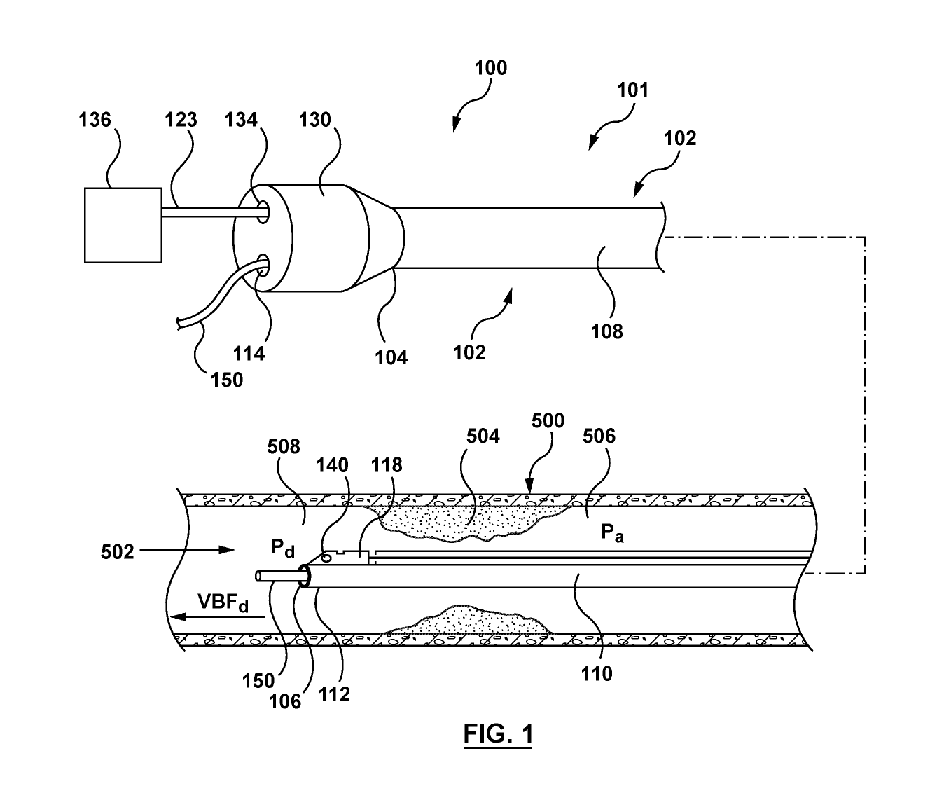

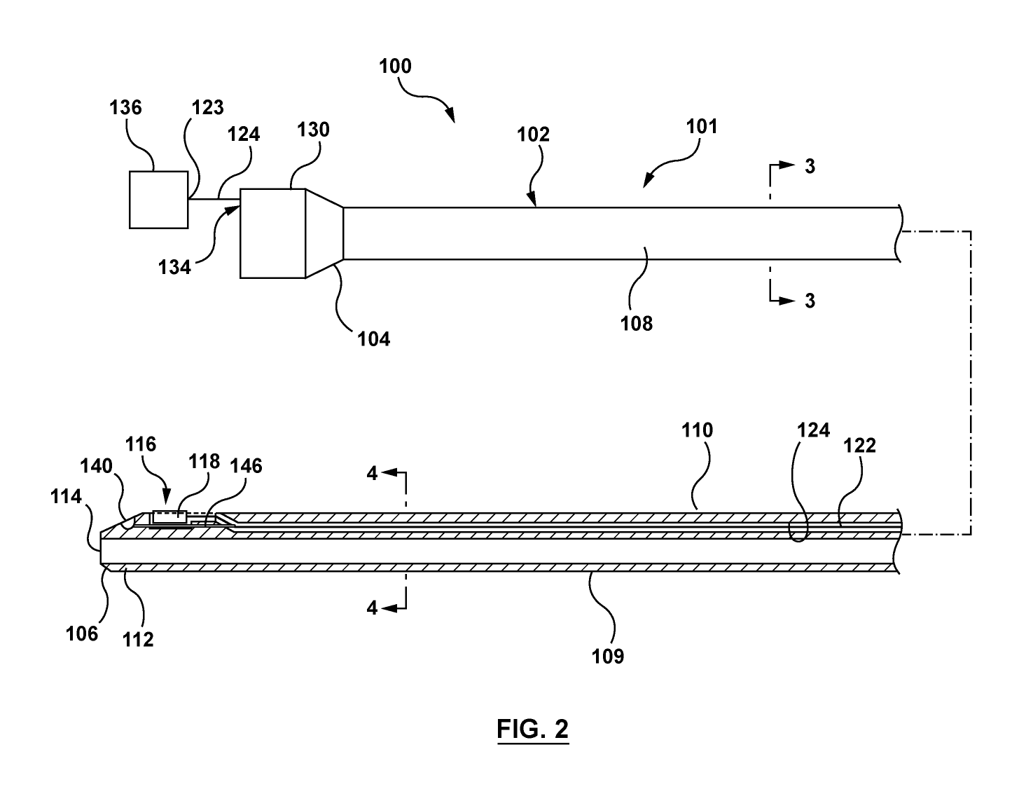

A system 100 for calculating a corrected Fractional Flow Reserve (FFR_{CORR}) according to an embodiment of the present disclosure is shown in FIG. 1 and in greater detail in FIGS. 2-7. System 100 includes a catheter 101, a proximal pressure-sensing device (not shown in FIGS. 1-7), and a processing device 136, as shown in FIG. 1. Catheter 101 includes a pressure sensor 118 and a thermistor 140 coupled thereto, as described in greater detail below. System 100 is configured to calculate a Fractional Flow Reserve (FFR) of a lesion 504 based on a distal pressure P_{d} relative to a proximal pressure Pₐ, as explained in greater detail below. System 100 is further configured to calculate or select a transfer function, including a flow velocity correction based on a measured blood flow velocity VBF_{d} distal of lesion 504. The transfer function is used to calculate the corrected FFR (FFR_{CORR}). Various features of the components of system 100 reflected in FIGS. 1-7 and described below can be modified or replaced with different structures and/or mechanisms. System 100 is merely an exemplary embodiment of a system for measuring and calculating FFR and FFR_{CORR} according to an embodiment hereof and modifications can be made to the embodiments described herein without departing from the spirit and scope of the present disclosure. The present disclosure is in not limited to the components described herein, and the components of system 100 may assume different forms and construction based upon application. Therefore, the following detailed description is not meant to be limiting.

Catheter 101 of system 100 may be a catheter or micro-catheter, and may include an elongate shaft 102 having a proximal end 104 coupled to a handle or luer 130, and a distal end 106, as shown in FIG. 1. Catheter 101 is configured to be disposed with a proximal portion 108 of elongate shaft 102 extending outside of a patient and a distal portion 110 of elongate shaft 102 positioned in situ within a lumen 502 of a vessel 500 having stenosis or lesion 504. In an embodiment, vessel 500 is a blood vessel such as, but not limited to, a coronary artery. Lesion 504 is generally representative of any blockage or other structural arrangement that results in a restriction to the flow of fluid through lumen 502 of vessel 500. Lesion 504 may be a result of plaque buildup, including without limitation plaque components such as fibrous, fibro-lipidic (fibro fatty), necrotic core, calcified (dense calcium), blood, fresh thrombus, and mature thrombus. Generally, the composition of lesion 504 will depend on the type of vessel being evaluated. In that regard, it is understood that embodiments hereof are applicable to various types of blockages or other narrowing of a vessel that results in decreased fluid flow.

Elongate shaft 102 includes proximal end 104, distal end 106, proximal portion 108, and distal portion 110, and defines a guidewire lumen 114 therein, as shown in FIG. 2. Elongate shaft 102 may be formed of a polymeric material, non-exhaustive examples of which include polyethylene, PEBA, polyamide and/or combinations thereof, either blended or co-extruded. Optionally, elongate shaft 102 or some portion thereof may be formed as a composite having a reinforcement material incorporated within a polymeric body in order to enhance strength and/or flexibility. Suitable reinforcement layers include braiding, wire mesh layers, embedded axial wires, embedded helical or circumferential wires, and the like. In one embodiment, for example, at least the proximal portion 108 of elongate shaft 102 may be formed from a reinforced polymeric tube. In other embodiments of an elongate tubular shaft or component in accordance herewith, a proximal segment thereof may be a hypotube of a medical grade stainless steel with outer and inner tubes of a distal segment thereof being formed from any of the polymeric materials listed above. Further, although proximal portion 108 and distal portion 110 of elongate shaft 102 are described separately, they are described in such a manner for convenience and elongate shaft 102 may be constructed unitarily such that the portions described are part of a unitary shaft. However, different portions of elongate shaft 102 may also be constructed separately and joined together.

While FIGS. 1-7 show elongate shaft 102 of catheter 101 with guidewire lumen 114 extending therethrough in an over-the-wire configuration, this is not meant to limit the design and other configurations may be utilized, including, but not limited to, a rapid exchange configuration, or other configurations suitable for the purposes described herein.

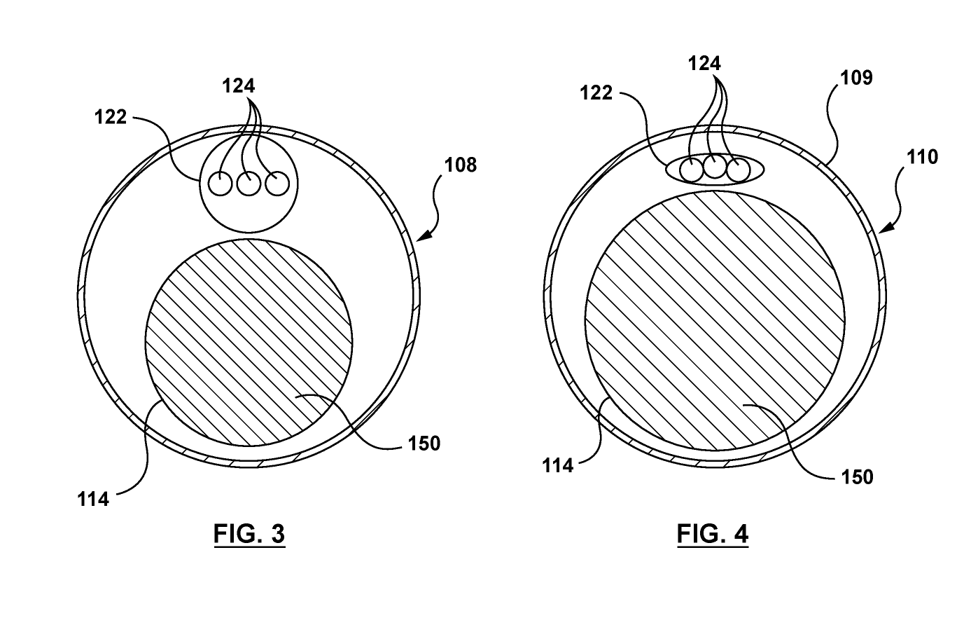

In an embodiment, proximal portion 108 of elongate shaft 102 is disposed proximal of distal portion 110 and extends proximally thereof to proximal end 104 of elongate shaft 102, as shown in FIG. 2. In an embodiment, proximal portion 108 defines two lumens, a proximal portion of guidewire lumen 114 (FIG. 3) and a proximal portion of a sensor wire lumen 122 (FIG. 3). In an embodiment, guidewire lumen 114 and sensor wire lumen 122 may be disposed parallel or side-by-side to each other along proximal portion 108, as shown in FIG. 3, taken along line 3-3 of FIG. 2.

Guidewire lumen 114 is configured to retain a guidewire 150 therein, as shown in FIGS. 3-4. In an embodiment, guidewire lumen 114 may be disposed from proximal end 104 of elongate shaft 102 to distal end 106 of elongate shaft 102, as shown in FIG. 2. Stated another way, guidewire lumen 114 may extend the length of elongate shaft 102, including proximal portion 108 and distal portion 110. Guidewire lumen 114 is sized to accept guidewire 150 therein. However, as explained above, guidewire lumen 114 may extend only along a portion of elongate shaft 102 in a rapid-exchange configuration. For example, and not by way of limitation, guidewire lumen 114 may be eliminated in proximal portion 108 of elongate shaft 102 such that the guidewire extends alongside a modified proximal portion of elongate shaft 102 that includes only sensor wire lumen 122. Further, while proximal portion 108 of elongated shaft 102 is shown with two lumens therein, this is not meant to limit the design and, and more or fewer lumens may be utilized.

Sensor wire lumen 122 is configured to retain a wire bundle 124 therein, as shown in FIGS. 3-4. In an embodiment, sensor wire lumen 122 is in communication with pocket 116 of distal portion 110, as shown in FIGS. 2, 5, and 6 and described in greater detail below. In an embodiment, sensor wire lumen 122 extends from pocket 116 to proximal end 104 of elongate shaft 102.

While sensor wire lumen 122 is depicted in FIG. 3 as circular in cross-section, this is not meant to limit the design, and other configurations may be utilized, including but not limited to elliptical, crescent-shaped, or other configurations suitable for the purposes described herein.

While FIGS. 1-6 include sensor wire lumen 122, this is not meant to limit the design, and other configurations may be utilized. For example, wire bundle 124 may extend from pressure sensor 118 and thermistor 140 other than in a sensor wire lumen. For example, and not by way of limitation, sensor wire lumen 122 may be eliminated and signals from pressure sensor 118 and thermistor 140 may be sent to processing device 136 via wireless transmission. In another non-limiting example, wire bundle 124 may be integrated into the wall of distal portion 110 and proximal portion 108 of elongate shaft 102.

Distal portion 110 of elongate shaft 102 is disposed distal of proximal portion 108 extending distally thereof to distal end 106 of elongate shaft 102, as shown in FIG. 2. In an embodiment, distal portion 110 includes a shaft wall 109 defining a portion of guidewire lumen 114 extending therethrough, as shown in FIG. 2 and FIG. 4, taken along line 4-4 of FIG. 2. Distal portion 110 further includes pressure sensor 118 disposed within pocket 116, thermistor 140, a distal tip 112, and a distal portion of sensor wire lumen 122, as shown in FIG. 2 and described in greater detail below. Distal portion 110 is configured to receive guidewire 150 in the distal portion of guidewire lumen 114, as shown in FIG. 4. Distal portion 110 is further configured, as shown in FIG. 1, to extend from a proximal side 506 of lesion 504, crossing (or extending past) lesion 504, to a distal side 508 of lesion 504 such that distal tip 112 is disposed on distal side 508 of lesion 504.

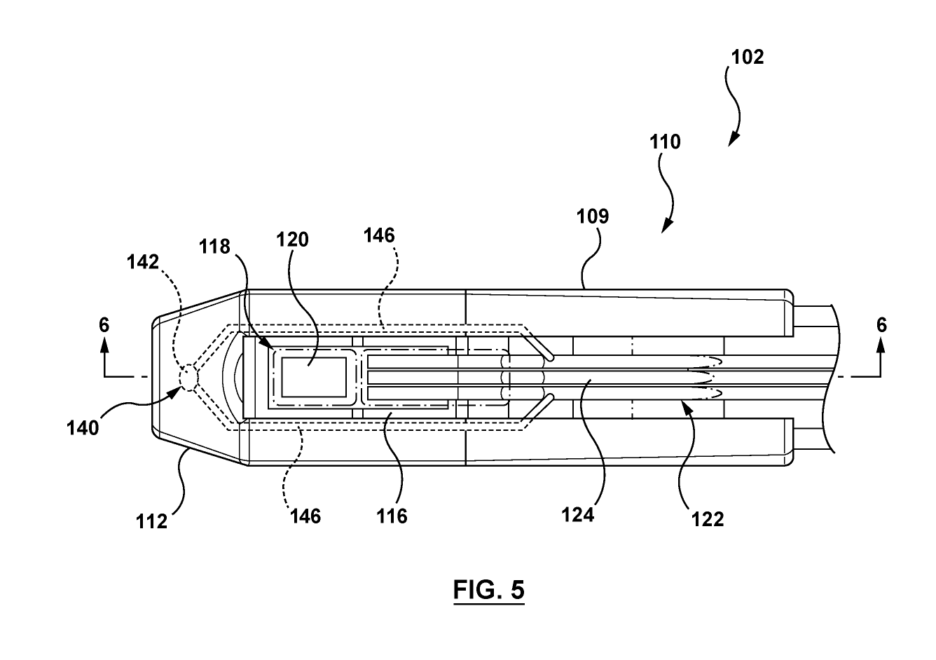

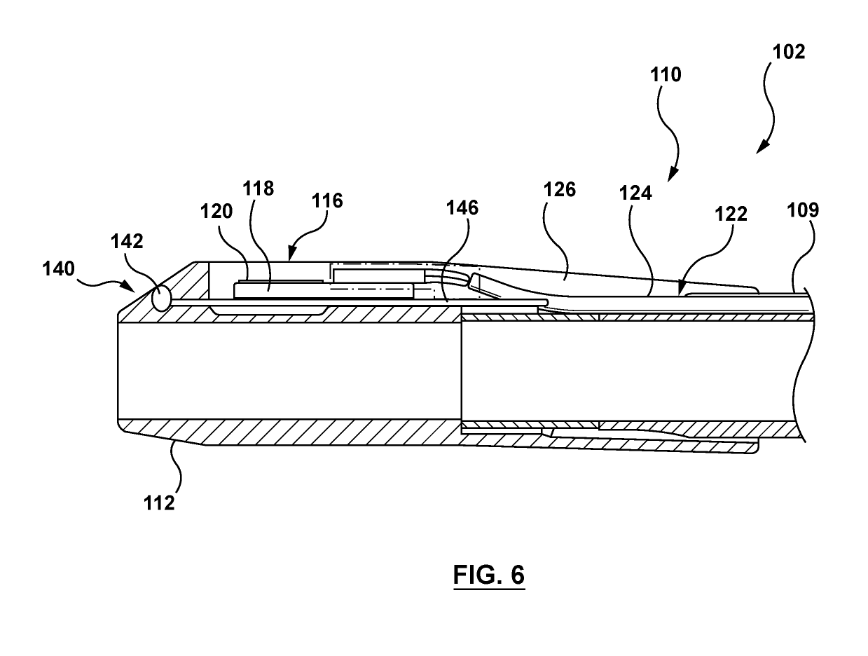

Pressure sensor 118 of distal portion 110 of elongate shaft 102 includes a pressure-sensing surface 120, as shown in FIGS. 5-6. Pressure sensor 118 may be a piezo-resistive pressure sensor, a piezo-electric pressure sensor, a capacitive pressure sensor, an electromagnetic pressure sensor, an optical pressure sensor, and/or combinations thereof suitable for the purposes described herein. Pressure sensor 118 is configured with pressure-sensing surface 120 facing outwardly from an outer surface of shaft wall 109 of distal portion 110 of elongate shaft 102 such that pressure-sensing surface 120 measures pressure P_{d} of a fluid outside distal portion 110 of elongate shaft 102. Pressure sensor 118 is further configured to communicate measured pressure P_{d} with processing device 136 (FIG. 1) through wire bundle 124, as shown in FIG. 2. In an embodiment, pressure sensor 118 is disposed within distal portion 110 of elongate shaft 102 and proximal of distal tip 112 such that pressure sensor 118 is disposed on distal side 508 of lesion 504, as shown in FIG. 1.

In an embodiment shown in FIGS. 5-6, pressure sensor 118 is disposed in and coupled to pocket 116 of elongate shaft 102. Pressure sensor 118 is further coupled to wire bundle 124. Pressure sensor 118 may be coupled to pocket 114, for example, and not by way of limitation, by adhesives, fusing, welding, or any other method suitable for the purposes of the present disclosure. Pressure sensor 118 may be coupled to wire bundle 124 for example, and not by way of limitation, by soldering, fusing, welding, for any other method suitable for the purposes of the present disclosure.

While pocket 114 is shown with a generally cubic shape, this is not meant to limit the design and other configurations of pocket 114 may be utilized, including, but not limited to spheroid, arched, or other configurations suitable for the purposes described herein. Further, pocket 114 and pressure sensor 118 may be configured with tabs, arms, slots, or other configurations suitable to enhance coupling of pressure sensor 118 with pocket 114. Further, other configurations for mounting pressure sensor 118 to distal portion 110 may be utilized. For example, and not by way of limitation, pressure sensor 118 may be coupled to distal portion 110 described in U.S. Patent Application Publication Nos. 2015/0305633 A1; 2015/0359438 A1; 2016/0081564 A1; and 2016/0199003 A1, each of which is incorporated by reference herein in its entirety.

Thermistor 140 coupled to distal portion 110 of elongate shaft 102 may be any thermistor suitable for the purposes described herein. In an embodiment shown in FIGS. 5-6, thermistor 140 is a bead-type thermistor. However thermistor 140 may be other thermistor types including, but not limited to rod, plate, disc, chip, or other configurations suitable for the purposes described herein. Thermistor 140 is coupled to distal portion 110 of elongate shaft 102 such that a surface 142 faces outside an outer surface of distal portion 110. Thus, when catheter 101 is disposed at the treatment site, as shown in FIG. 1, surface 142 of thermistor 140 is exposed to blood flow on distal side 508 of lesion 504. In one non-limiting example thermistor 140 is about 0.15 mm to 0.5 mm in diameter. However, other sized thermistors with other shapes may be utilized. Thermistor 140 may be coupled to distal portion 110, for example, and not by way of limitation, by adhesives, fusing, welding, for any other method suitable for the purposes of the present disclosure. Thermistor 140 is further coupled to wire bundle 124. Thermistor 140 may be coupled to wire bundle 124 for example, and not by way of limitation, by soldering, fusing, welding, for any other method suitable for the purposes of the present disclosure.

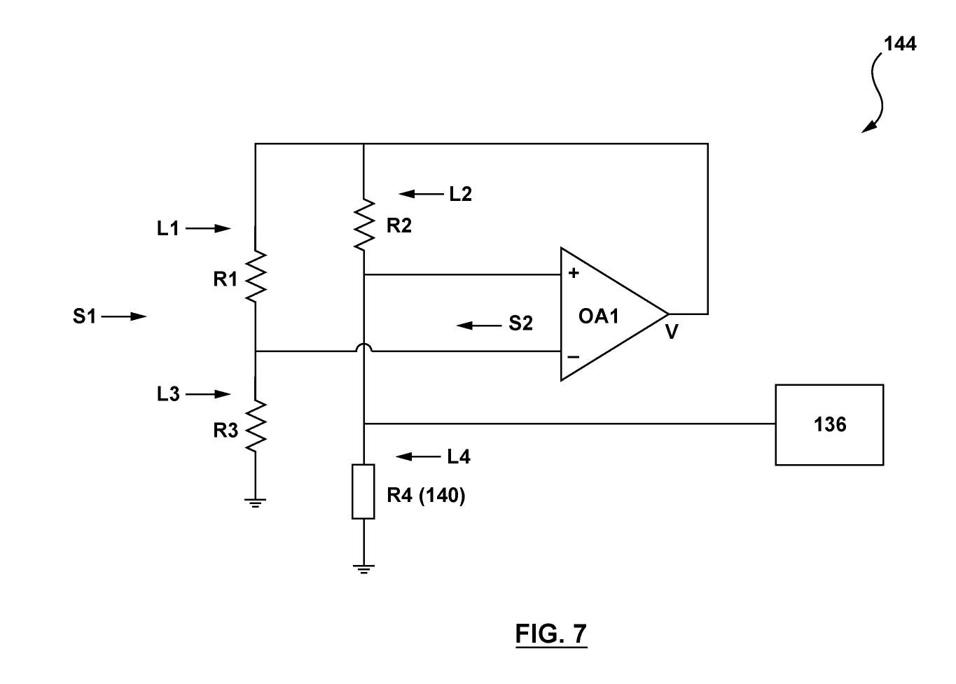

Thermistor 140 forms a leg of a Wheatstone bridge 144, an embodiment of which is shown in FIG. 7. Wheatstone bridge 144 is also known as a null comparator. Wheatstone bridge 144 may be any Wheatstone bridge design suitable for the purposes described herein. In the embodiment shown, Wheatstone bridge 144 includes two (2) sides S1 and S2 and an operational amplifier OA1, as shown in FIG. 7. Side S1 includes two (2) legs L1 and L3. Side S2 includes two (2) legs L2 and L4. Each leg L1, L2, L3, and L4 includes a corresponding resistor R1, R2, R3, and R4. Wheatstone bridge 144 is configured with thermistor 140 as resistor R4 of leg L4. Thermistor 140 includes a resistive value R4 that changes based on temperature. At a predetermined temperature, such as 1-2° C. above body temperature, the resistive value R4 of thermistor 140 balances Wheatstone bridge 144. The function of Wheatstone bridge 144 is described in greater detail below.

In an embodiment, thermistor 140 of Wheatstone bridge 144 is disposed at distal portion 110 of catheter 101, and the remainder of Wheatstone bridge 144 is disposed at processing device 136. However, other configurations may be utilized. For example, and not by way of limitation, all of Wheatstone bridge 144 may be disposed at distal portion 110. In another non-limiting example, thermistor 140 may be disposed at distal portion 110, with the remainder of Wheatstone bridge 144 as a stand-alone device in communication with processing device 136. Other configurations suitable for the purposes described herein may also be utilized.

Processing device 136, as shown in FIGS. 1-2, may be any processing device suitable for the purposes described herein. Processing device 136 may include such components as a CPU, a display device, an amplification and filtering device, an analog-to-digital converter, portions of Wheatstone bridge 144, and various other components. Processing device 136 is configured to receive a proximal pressure Pₐ and a distal pressure P_{d}. Processing device 136 is further configured to monitor power draw of thermistor 140 of Wheatstone bridge 144, as explained in more detail below. Processing device 136 is further configured to process Pₐ, P_{d}, and thermistor power measurements as described in greater detail below, and provide a continuous display of FFR_{corr}. Processing device 136 is coupled to wire bundle 124 such that processing device 136 is in communication with pressure sensor 118. Processing device 136 is further in communication with Wheatstone bridge 144. A proximal end 123 of wire bundle 124 and Wheatstone bridge 144 may be coupled to processing device 136 via various communication pathways, including but not limited to one or more physical connections including electrical, optical, and/or fluid connections, a wireless connection, and/or combinations thereof. Accordingly, it is understood that additional components (e.g., cables, connectors, antennas, routers, switches, etc.) not illustrated in FIG. 1 and FIG. 7 may be included to facilitate communication between proximal end 123 of wire bundle 124 and processing device 136, and between Wheatstone bridge 144 and processing device 136. In other embodiments, instead of a dedicated sensor wire lumen 122, communication between pressure sensor 118 and processing device 136 may be accomplished wirelessly, or wire bundle 124 may be incorporated into the wall of elongate shaft 102.

In an embodiment, as shown in FIG. 2, pocket 116 is in communication with sensor wire lumen 122 such that wire bundle 124 from pressure sensor 118 may extend from pocket 116 proximally through sensor wire lumen 122, through a corresponding lumen or luer 130 exiting through a proximal port 134 to processing device 136 coupled to proximal end 123 of wire bundle 124. Additionally, thermistor 140 is in communication with sensor wire lumen 122 such as through wires 146 (FIG. 5). Wires 146 may connect thermistor 140 to wire bundle 124, which extends proximally through sensor wire lumen 122, through a corresponding lumen (not shown) in luer 130 exiting through proximal port 134 to the remainder of Wheatstone bridge 144 coupled to proximal end 123 of wire bundle 124. In the embodiment of FIGS. 5-6, wire bundle 124 is utilized to connect both pressure sensor 118 and thermistor 140 utilizing a multiplexed signal over wire bundle 124 to processing device 136 and Wheatstone bridge 144.

With an understanding of the components of system 100 above, the interactions of the various components, inputs, and calculations of system 100 will be described. System 100 is configured to determine FFR_{CORR} of a vessel. Distal portion 110 of elongate shaft 102 is configured to cross lesion 504 such that pressure sensor 118 and thermistor 140 are disposed on distal side 508 of lesion 504, as shown in FIG. 1.

As explained above, thermistor 140 is disposed on distal portion of elongate shaft 102 such that thermistor 140 is exposed to blood flow on distal side 508 of lesion 504. Thermistor 140 is configured to measure blood flow on distal side 508 of lesion 504. Thus, as explained above, thermistor 140 is configured such that Wheatstone bridge 144 is in a balanced configuration when thermistor 140 is at a predetermined temperature over body temperature. Therefore, when balanced, the following relationships apply.

Balanced configuration: R1/R3=R2/R4 (1)

Ohm's Law: V=IR or V/R=I (2)

(wherein V is voltage, I is current, and R is resistive value)

Power: P=VI (3)

Therefore, power P is directly proportional to voltage V and current I. Substituting equation 2 into equation 3:

P=V²/R (4)

Therefore, power P is inversely proportional to resistance R. Stated another way, as resistance R decreases, power P increases.

P∝1/R (5)

Thermistor 140 is further configured such that as blood flow BF_{d} of distal side 508 of lesion 504 (FIG. 1) contacts surface 142 (FIG. 5) of thermistor 140, resistance R4 of thermistor 140 decreases. With resistance R4 decreased, Wheatstone bridge 144 transitions to an unbalanced configuration. To balance Wheatstone bridge 144, side S2 (with thermistor 140) will draw additional current ΔI as defined by Ohm's Law. As current I is directly proportional to power P, side S2 will draw additional power ΔP. Therefore, when Wheatstone bridge 144 is in the unbalanced configuration, the following relationships apply:

Unbalanced configuration: R1/R3≠R2/R4 (6)

Power: P=V²/R (7)

Therefore, with voltage V constant, as resistance R4 (thermistor 140) decreases, power P increases. Accordingly, processing device 136 is configured to measure ΔP of Wheatstone bridge based on the change in resistance R4 of thermistor 140.

Additional power(Side 2): ΔP=P_{unbalanced}−P_{balanced} (8)

Processing device 136 is further configured to calculate blood flow velocity VBF_{d} from ΔP. In particular, the ability of a medium (such as blood) to dissipate heat may be defined as:

P/dT=ability of medium to dissipate heat (9)

(where P is power and dT is the temperature difference across thermistor 140 and blood flow BF). Heat dissipation is proportional to blood temperature, thermal properties, and blood flow velocity V_{BFd}.

Power P, specifically ΔP, is measured by processor 136, as described above. Temperature difference dT is assumed to be constant. Blood temperature is assumed to be constant. Thermal properties of blood flow BF are assumed to be constant. Therefore, power P, or additional power ΔP, is proportional to blood flow velocity V_{BFd}:

ΔP∞V_{BFd} (10)

Thus, by measuring ΔP of Wheatstone bridge 144 caused by blood flow across thermistor 140, processing device 136 can calculate blood flow velocity. In particular, empirical data may be used to correlate ΔP to blood flow velocity. For example, and not by way of limitation, various blood flow velocities may be measured using conventional techniques when not performing an FFR measurement procedure. For example, and not by way of limitation, blood flow velocities may be measured using videocapillary microscoping with frame-to-frame analysis, or other techniques known to those skilled in the art. Simultaneously, a thermistor and a Wheatstone bridge, as described above, are used to obtain ΔP. Using the data comparing measured blood flow velocities to measured ΔP, a table may be created to correlate ΔP to blood flow velocity. Thus, blood flow velocities may be calculated from the table which correlates ΔP to blood flow velocity. Alternatively, an established look-up table correlating ΔP to blood flow velocities may be used. Thus, by obtaining ΔP from as described above, processing device 136 may utilize the established look-up table to obtain the blood flow velocity corresponding to ΔP.

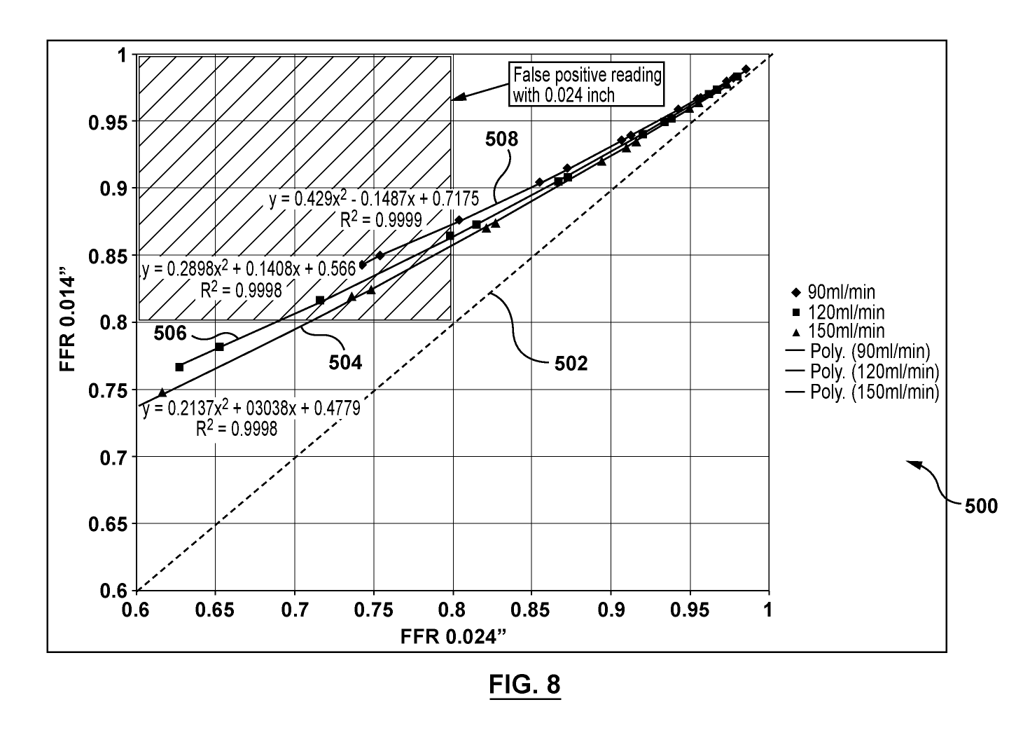

Processing device 136 is further configured to utilize blood flow velocity VBF_{d}, difference in catheter diameter (catheter diameter versus diameter of a standard FFR guidewire), and vessel diameter to determine transfer function coefficients to calculate a corrected FFR. Processing device 136 may take other information into account, such as, but not limited to, the length of the lesion, length of the blood vessel, etc. to calculate a corrected FFR. FIG. 8 shows a particular example of using a look up table in which the blood flow velocity VBF_{d} and difference in catheter diameter are used to establish a formula for correcting FFR.

Thus, referring to a graph 500 in FIG. 8, the horizontal axis represents FFR values for 0.024 inch FFR catheter. The vertical axis represents FFR values for a 0.014 inch FFR wire. If no difference existed between measuring FFR with a 0.024 in FFR catheter and a 0.014 inch FFR wire, measured date would fall generally on line 502. In other words, there would be a 1:1 correlation between measuring FFR with a 0.024 inch FFR catheter and a 0.014 inch FFR wire. However, the measurements do differ. Thus, in FIG. 8, experimental data is used to compare FFR measurements with a 0.024 inch catheter to FFR measurements with a 0.014 inch catheter. Further, the experimental data varies the blood flow rate. Thus, referring to FIG. 8, the diamond shaped data points are measured FFR for a 0.024 inch FFR catheter and a 0.014 inch FFR wire at a blood flow rate of 90 ml/minute. The square shaped dated points are measured FFR for a 0.024 inch FFR catheter and a 0.014 inch FFR wire at a flow rate of 120 ml/minute. The triangular shaped data points are measured FFR for a 0.024 inch FFR catheter and a 0.014 inch FFR wire at a flow rate of 150 ml/minute. Thus, for example, there is a square data point at 0.8 on the X-axis and just above 0.85 on the Y-axis. This data point represents that for a particular simulated lesion and a blood flow rate of 120 ml/minute, an FFR value of 0.8 measured using a 0.024 inch FFR catheter correlates to an FFR value of just above 0.85 if the FFR value had been measured with a 0.014 inch FFR wire.

As noted above, it is important to correlate to an FFR value measured with a 0.014 inch FFR wire because historical data on whether to perform an interventional treatment on a patient based on FFR value has been based on FFR value using an FFR wire. For example, historically, FFR values taken with a 0.014 inch FFR wire that are less than about 0.80 are generally deemed significant and in need of an interventional treatment. Thus, in the chart of FIG. 8, there is a shaded portion that is a “false positive” area. This is based on the 0.80 FFR value. Thus, the shaded area represents an area that a 0.024 inch FFR catheter measures an FFR value of less than or equal to 0.80, thereby indicating intervention, but that the measured FFR value using a 0.014 inch FFR wire would have been above 0.80, thereby not indicating intervention.

The different data points for each flow rate are for different lesions, which is why there are several data points for each flow rate. FIG. 8 is only an example, and other flow rates may be included, such as in 10 ml/minute increments. Further, while FIG. 8 correlates FFR taken with a 0.024 inch FFR catheter with FFR taken with a 0.014 in FFR wire, this is not meant to be limiting. Thus, similar charts and formulas (discussed below) for other sizes of FFR catheters, such as but not limited to a 0.020 inch FFR catheter, may be used. With the data points, line 504 is a best fit line through the data for the 150 ml/minute flow rate, line 506 is the best fit line through the data for the 120 ml/minute data points, and line 508 is the best fit line through the data for the 90 ml/minute line. Above each line is a formula or transfer function for correlating the FFR measured using a 0.024 inch FFR catheter to the FFR value of the same lesion at the same flow rate measured using a 0.014 inch FFR wire. This formula or transfer function is how the FFR measured using a 0.024 inch FFR catheter is corrected or correlated to FFR measured using a 0.014 inch FFR wire. The formulas take the form of:

Y=aX²+bX+c (11)

where Y is the corrected FFR, X is the measured FFR using a 0.024 inch FFR catheter at the given flow rate, and “a”, “b”, and “c” are transfer function coefficients that are determined by finding the best fit through the data points. Thus, for example, for 120 ml/minute flow rate, the formula to correct FFR is as follows:

FFR_{Corr}=0.2898(FFR)²+0.1408(FFR)+0.566 (12)

Thus, in an example, if the thermistor results in a measured blood flow rate of 120 ml/minute, and the FFR measured with the 0.024 in FFR catheter is 0.80, the corrected FFR is as follows:

FFR_{Corr}=0.2898(0.80)²+0.1408(0.80)+0.566=0.864 (13)

With the above equations in mind, FFR and corrected FFR may be calculated. In particular, a distal pressure P_{d} of blood on distal side 508 of lesion 504 is sensed by pressure sensor 118 and is communicated with processing device 136. Simultaneously, an aortic or proximal pressure Pₐ of blood on proximal side 506 of lesion 504 is sensed and communicated with processing device 136. Aortic pressure Pₐ may be sensed by an aortic or proximal pressure measurement device (not shown). The aortic pressure measurement device may be part of catheter 101 or may be a separate device. Generally, the aortic pressure measurement device is part of a guide catheter inserted into the aorta with an external AO pressure transducer. However, other devices can be used to for measuring the aortic or proximal pressure.

Processing device 136 calculates FFR using the measured proximal and distal pressures Pₐ and P_{d}, as shown in equation 13 below. Processing device 136 also calculates the blood flow rate as discussed above. Based on the blood flow rate, processing device 136 determines the appropriate formula/transfer function to calculate the corrected FFR. Processing device 136 then uses the calculated FFR and the transfer function to calculate FFR_{Corr}. In particular, the following calculations are used to obtain the FFR_{Corr} value.

FFR=P_{d}/Pₐ (14)

FFR_{Corr}=a(FFR)²+b(FFR)+c (15)

The coefficients “a”, “b”, and “c” are based on empirical data using the catheter size and blood flow rate. Other factors, such as lesion length, vessel diameter, etc. may also be taken into account using empirical data as outlined above with respect to FIG. 8.

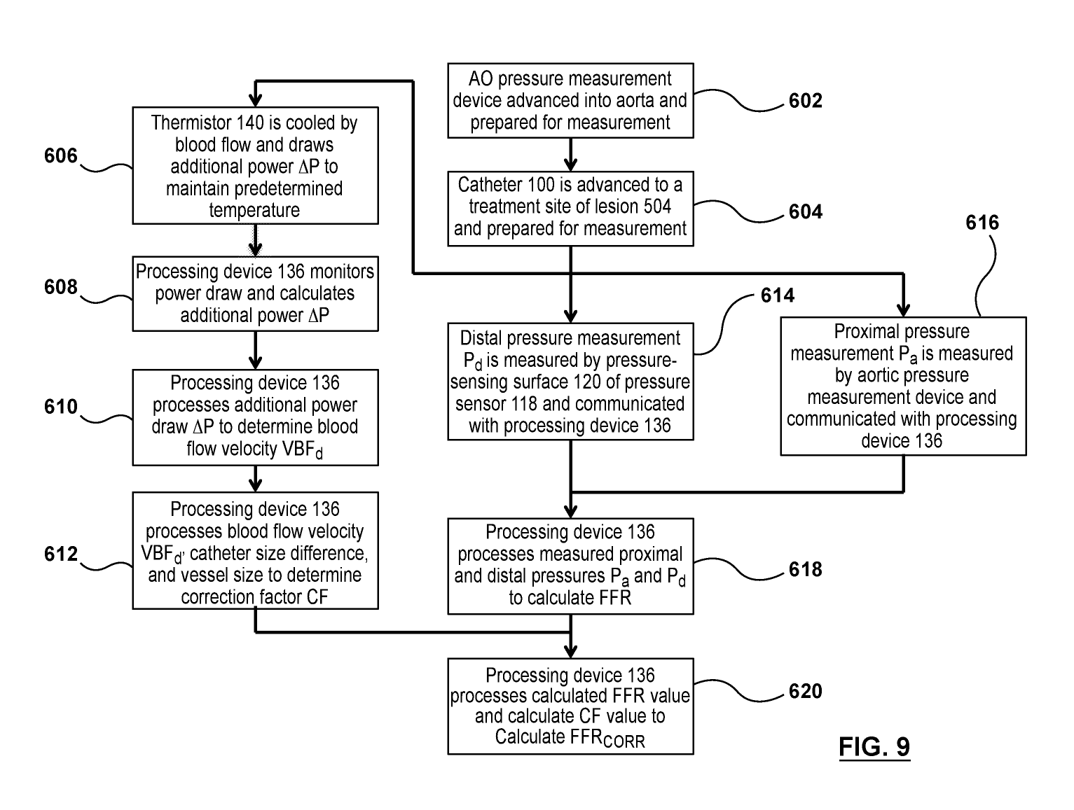

FIG. 9 shows an embodiment of a method of calculating a corrected Fractional Flow Reserve (FFR_{Corr}). Steps 602-620 of FIG. 9 reference components and the vasculature shown in FIG. 1.

In step 602, an aortic pressure measurement device (not shown) is advanced within the vasculature. In a non-limiting example, the aortic pressure measurement device may be a guide catheter (not shown) with an external pressure transducer (not shown), as known in the art. However, this is not meant to be limiting, and other proximal pressure measurement devices may be utilized. The proximal pressure measure measurement device is configured to measure proximal pressure Pₐ.

In step 604 of the embodiment of the method of FIG. 9, catheter 101 is advanced into a patient's vasculature, and positioned such that pressure sensor 118 and thermistor 140 are positioned on distal side 508 of lesion 504. Radiopaque markers may be utilized to determine when distal portion 110 of catheter 101 is in the proper position. In an embodiment, catheter 101 is advanced through a guide catheter (not shown).

In step 606 of the embodiment of the method of FIG. 8, with catheter 101 in position, surface 142 of thermistor 140 is cooled by blood flow BF_{d} on distal side 508 of lesion 504. As thermistor 140 cools from a predetermined temperature above body temperature, thermistor 140 draws additional power to balance the Wheatstone bridge 144.

In step 608 of the embodiment of the method of FIG. 9, processing device 136 monitors power draw and calculates additional power draw ΔP required to maintain Wheatstone bridge 144 balanced. In step 610 of the embodiment of FIG. 9, processing device 136 processes the additional power draw ΔP to calculate blood-flow velocity VBF_{d} (VBF_{d} is proportional to additional power draw ΔP of thermistor 140), as explained above.

In step 612 of the embodiment of FIG. 9, processing device 136 processes blood flow velocity VBF_{d}, catheter size difference (catheter to FFR guidewire), and vessel size to determine transfer function coefficients, and uses the transfer function coefficients to calculate corrected FFR, as explained above.

Simultaneous with step 608, in step 614 of the embodiment of FIG. 9, distal pressure P_{d} is measured by pressure-sensing surface 120 of pressure sensor 118 on distal side 508 of lesion 504. Distal pressure P_{d} is communicated to processing device 136.

Simultaneous with steps 608 and 614, in step 616 of the embodiment of the method of FIG. 9, proximal pressure Pₐ of blood on proximal side 506 of lesion 504 is measured by the aortic pressure measurement device (not shown), such as an external AO pressure transducer. Measured proximal pressure Pₐ is communicated to processing device 136.

In step 618 of the embodiment of the method of FIG. 9, processing device 136 processes measured proximal and distal pressures Pₐ and P_{d} to calculate FFR. In step 620, processing device 136 processes the calculated FFR value and calculated transfer function coefficients to calculate FFR_{CORR}.

While the method of FIG. 9 shows proximal pressure Pₐ as measured by an external AO pressure transducer, this is not meant to limit the method, and other methods of obtaining proximal pressure Pₐ may be utilized, including replacing the step of measuring proximal pressure Pₐ with external AO pressure transducer of step 616 with a step of measuring proximal pressure Pₐ with a pressure-sensing device coupled to catheter 101, measuring proximal pressure Pₐ with pressure sensor 118 either before or after the step of measuring distal pressure P_{d} with pressure sensor 118, or measuring proximal pressure Pₐ by any other method suitable for the purposes described in step 616.

Although the method of FIG. 9 is described with respect to system 100 of FIG. 1 in coronary artery 510, those skilled in the art will understand that the method described herein may utilize a system according to any embodiment described herein, and may be used in other bodily vessels where a FFR_{CORR} measurement is useful.

While the embodiments and methodology of FIGS. 1-9 describe a thermistor coupled to a catheter to calculate FFR_{CORR}, this is not meant to limit the design and method, a thermistor disposed on a guidewire-based FFR system may be utilized with a similar methodology to FIG. 9 to calculate FFR_{CORR} for blood flow velocity.

While only some embodiments according to the present invention have been described herein, it should be understood that they have been presented by way of illustration and example only, and not limitation. Various changes in form and detail can be made therein without departing from the spirit and scope of the invention. Further, each feature of each embodiment discussed herein, and of each reference cited herein, can be used in combination with the features of any other embodiment. All patents and publications discussed herein are incorporated by reference herein in their entirety.

What is claimed is:

1. A catheter comprising:
an elongate shaft defining a guidewire lumen;
a pressure sensor coupled to a distal portion of the elongate shaft such that a pressure-sensing surface of the pressure sensor faces outside of an outer surface of the distal portion of the elongate shaft; and
a thermistor coupled to the distal portion of the elongate shaft such that a surface of the thermistor faces the outside of the outer surface of the distal portion;
wherein the pressure sensor is configured to sense a pressure of blood distal of a stenosis in a blood vessel, and wherein the pressure sensor is in communication with a processor;
wherein the thermistor is configured to measure a blood flow velocity distal of the stenosis such that the processor may calculate a corrected Fractional Flow Reserve including a correction based on the blood flow velocity.

2. The catheter of claim 1, wherein the thermistor is one arm of a Wheatstone bridge.

3. The catheter of claim 2, wherein the blood flow velocity is proportional to power required to maintain balance of the Wheatstone bridge, wherein the thermistor changes resistance based on exposure to blood flow, and wherein the change in resistance unbalances the Wheatstone bridge.

4. A system for calculating a corrected Fractional Flow Reserve associated with a stenosis in a blood vessel comprising:
a pressure sensor configured for placement within a blood vessel to measure a distal pressure distal to the stenosis;
a pressure-sensing device configured to measure a proximal pressure proximal of the stenosis;
a thermistor configured for placement within the blood vessel to measure blood flow velocity distal of the stenosis; and
a processing device in communication the pressure sensor, the pressure-sensing device, and the thermistor;
wherein the processing device is configured to calculate a Fractional Flow Reserve based on the distal pressure relative to the proximal pressure; and
wherein the processing device is configured to calculate or select a transfer function including a flow velocity correction based on the measured blood flow velocity, wherein the processing device is configured to apply the transfer function to the calculate Fractional Flow Reserve to calculate a corrected Fractional Flow Reserve.

5. The system of claim 4,
wherein the thermistor is one arm of a Wheatstone bridge,
wherein power to maintain the thermistor at a predetermined temperature is proportional to blood flow velocity, and
wherein the processor is configured to calculate blood flow velocity based on the power to maintain the thermistor at the predetermined temperature.

6. The system of claim 4,
wherein the pressure sensor is coupled to a catheter having a catheter cross-sectional area in a portion of the catheter configured to cross the stenosis to measure the distal pressure, and
wherein the transfer function selected or calculated by the includes a size correction based on the catheter cross-sectional area as compared to a cross-sectional area of a wire-based pressure-sensing device.

7. The system of claim 6,
wherein the transfer function selected or calculate by the processor includes a vessel size correction based on a size of the blood vessel at the stenosis.

8. A method for calculating a corrected Fractional Flow Reserve in a vessel, the method comprising the steps of:
delivering a catheter to a distal side of a stenosis of the vessel, the catheter including a distal pressure sensor and a thermistor;
measuring the blood flow velocity within the vessel using the thermistor;
calculating or selecting a transfer function including a blood flow velocity correction based on the measured blood flow velocity;
measuring a pressure distal of the stenosis;
measuring a pressure proximal of the stenosis; and
calculating the corrected Fractional Flow Reserve using the measured proximal and distal pressures and application of the transfer function.

9. The method of claim 8, wherein the blood vessel is a coronary artery.

10. The method of claim 8, wherein the thermistor is one arm of a Wheatstone bridge, and wherein blood flow velocity is measured based on power to maintain the thermistor at a predetermined temperature.

11. The method of claim 8, wherein the transfer function also includes a catheter-size correction, wherein the catheter-size correction is selected or calculated based on a cross-sectional area of the catheter in a portion of the catheter configured to cross the lesion.
